(19) United States
(12) Patent Application Publication
(10) Pub. No.: US 2012/0252052 A1
(43) Pub. Date: Oct. 4, 2012

(54) METHOD FOR MULTIPLE QUANTIFICATION OF AMINO GROUP-CONTAINING NON-PEPTIDIC COMPOUND WITH HIGH EFFICIENCY AND HIGH SENSITIVITY AND KIT THEREFOR

(75) Inventors: Shigeru MATSUKAWA, Fukui (JP); Kazumi NARITA, Fukui (JP); Haruki SHIMODAIRA, Tokyo (JP)

(73) Assignees: Taiyo Nippon Sanso Corporation, Tokyo (JP); National University Corporation University of Fukui, Fukui-shi (JP)

(21) Appl. No.: 13/436,393

(22) Filed: Mar. 30, 2012

(30) Foreign Application Priority Data
Mar. 31, 2011 (JP) 2011-080943
Feb. 22, 2012 (JP) 2012-036598

(51) Int. Cl. C12Q 1/02; C07D 213/20; C07C 211/48

(52) U.S. Cl. 435/29; 564/305; 546/347

(57) ABSTRACT

A method of quantifying a target non-peptidic compound having an amino group contained in one or more biological samples, which comprises a step of producing a difference in the mass of the target non-peptidic compound between samples, by using a combination of two or more kinds of stable isotopes of a compound represented by the formula (I):

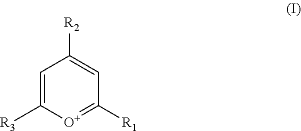

wherein R₁, R₂ and R₃ are the same or different and each is hydrogen, halogen or alkyl, or a salt thereof, as a labeling compound; and a kit and the like usable for such method.

### TECHNICAL FIELD

The present invention relates to a method for quantifying an amino group-containing non-peptidic compound contained in a biological sample and a kit therefor, and the like. More particularly, the present invention relates to a method for multiple quantification of an amino group-containing non-peptidic compound to be the analysis target such as biologically active amine, amino acid or the like, which is contained in each of one or more biological samples to be analyzed, by using a mass spectrometer, a kit that can be used for the method and the like.

### BACKGROUND OF THE INVENTION

Conventional analysis of amino acid employs a quantification method including mutual separation of 20 or more kinds of biological amino acids by one performance of high performance liquid chromatography (HPLC) analysis, reacting the amino acids with ninhydrin, and quantifying the amino acids based on the absorbances of the resultant products. However, the sensitivity of this method is low, and is not suitable for the analysis of an amino acid having a low concentration.

To achieve higher sensitivity, therefore, use of a fluorescent label has been proposed, which realizes about 10-fold or more higher sensitivity.

Furthermore, for an amino acid having a low concentration that cannot be detected even thereby, a method including separation by liquid chromatography, and subsequent detection by a mass spectrometer is adopted. In this case, to facilitate ionization of separated amino acids, amino acids are derivatized using a reagent that reacts with an amino group, and then subjected to liquid chromatographic separation and mass spectrometry. However, even when this method is used, the conventional technique has a limitation in high sensitivity analysis.

One of the present inventors previously developed a method of quantifying a protein contained in two or more samples, which uses a mass spectrometer, comprising producing a difference in the mass of the same protein contained in each sample by using a combination of two or more kinds of stable isotopes of a compound represented by the following formula:

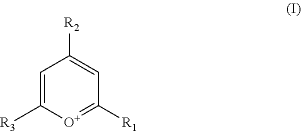

wherein R₁, R₂ and R₃ are the same or different and each is hydrogen, halogen or alkyl, or a salt thereof as a labeling compound (patent document 1). The technique disclosed in the document has overcome difficulty present in the actual application of protein quantification, which is caused by the problems of conventional techniques in functionality, efficiency, convenience, economic efficiency and the like.

Thus, although a superior technique of protein quantification was proposed by one of the present inventors, as for an amino group-containing non-peptidic compound such as amino acid, biologically active amine and the like, a technique capable of satisfying the current needs is absent at present as mentioned above.

### DOCUMENT LIST

### Patent Document

patent document 1: WO2008/156139

### SUMMARY OF THE INVENTION

### Problems to be Solved by the Invention

The above-mentioned current condition in the pertinent technical field requires a method capable of quantifying an amino group-containing non-peptidic compound in a biological sample with higher sensitivity. In addition, provision of a method capable of comprehensively analyzing biologically active amine, amino acid and the like present in each sample by multiple quantification of plural biological samples is extremely useful. The present invention provides such method. The present invention also provides a kit and the like usable for such method.

### Means of Solving the Problems

The present inventors have conducted intensive studies in an attempt to solve the above-mentioned problems. They have envisaged a methodology including adopting a nano-liquid chromatographic mass spectrometry system to achieve higher sensitivity, derivatizing an amino group-containing non-peptidic compound to be the analysis target with an amino reactive reagent that is positively charged in a pH-independent manner, thereby to improve ionization efficiency of the compound, and producing a difference in the mass of the compound between samples by using, in the reagent, a combination of stable isotopes, thereby to collectively analyze plural samples. They have found that analysis according to this methodology enables not only detection of an amino group-containing non-peptidic compound in a biological sample with high sensitivity, but also comprehensive analysis of an amino group-containing non-peptidic compound contained in each sample by simultaneous quantification of plural samples. They have also found that labeling of an amino group-containing non-peptidic compound in a biological sample with the above-mentioned amino reactive reagent affords a resultant product expected from a conventionally-known reaction pathway, as well as a resultant product having a different structure. The present inventors have conducted further studies based on these findings and completed the present invention.

Accordingly, the present invention provides the following.

[1] A method of quantifying a target non-peptidic compound having an amino group contained in one or more biological samples, which comprises the following steps:
(step 1) a step of preparing one or more biological samples to be analyzed, and an internal standard sample containing a known quantity of the target non-peptidic compound;
(step 2) a step of adding, as a compound indicative of mixing ratio, an amino group-containing non-peptidic compound, which is absent in the one or more biological samples and the internal standard sample, to each of the one or more biological samples and the internal standard sample, to a known concentration;
(step 3) a step of producing a difference in the mass of the target non-peptidic compound between samples comprising the one or more biological samples and the internal standard sample, and in the mass of the compound indicative of mixing ratio between samples comprising the one or more biological samples and the internal standard sample, by using a combination of two or more kinds of stable isotopes of a compound represented by the formula (I):

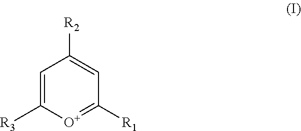

wherein R₁, R₂ and R₃ are the same or different and each is hydrogen, halogen or alkyl, or a salt thereof, as a labeling compound;
(step 4) a step of taking a given amount each from the one or more biological samples and the internal standard sample, and mixing them to give a mixture;
(step 5) a step of subjecting the mixture to mass spectrometry, and obtaining the intensities of peaks in a mass spectrum, which correspond to the masses of the target non-peptidic compounds having a mass difference from each other, and the intensities of peaks in the mass spectrum, which correspond to the masses of the compounds indicative of mixing ratio having a mass difference from each other;
(step 6) a step of determining a quantitative ratio between the target non-peptidic compounds having a mass difference from each other, which are contained in the mixture, based on an intensity ratio of the intensity corresponding to each of the target non-peptidic compounds derived from each of the one or more biological samples, and the intensity corresponding to the target non-peptidic compound derived from the internal standard sample; and
(step 7) a step of determining an absolute quantity of the target non-peptidic compound contained in each of the one or more biological samples, based on the quantitative ratio determined in step 6 and a mixing ratio of the samples determined by comparison of the intensities of peaks in the mass spectrum, which correspond to the masses of the compounds indicative of mixing ratio having a mass difference from each other.
[2] The method of the above-mentioned [1], wherein the compound of the formula (I) is 2,4,6-trimethylpyrylium.
[3] The method of the above-mentioned [1], wherein at least one of the target non-peptidic compounds and the compounds indicative of mixing ratio, from which the peak intensity obtained in step 5 is derived, is a compound represented by the formula (II):

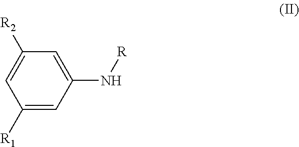

wherein R₁ and R₂ are as defined above, and R is any optionally substituted hydrocarbon group, excluding a compound having a peptide bond, or a salt thereof.
[4] The method of the above-mentioned [1], wherein the combination comprises 5 or more kinds of stable isotopes.
[5] The method of the above-mentioned [1], wherein the target non-peptidic compound is biologically active amine and/or amino acid.
[6] The method of the above-mentioned [5], wherein the biologically active amine is dopamine.
[7] The method of the above-mentioned [1], wherein the mass spectrometry is performed by a nano-liquid chromatographic mass spectrometer.
[8] A kit for quantifying a target non-peptidic compound having an amino group contained in one or more biological samples, which comprises a combination of two or more kinds of stable isotopes of a compound represented by the formula (I):

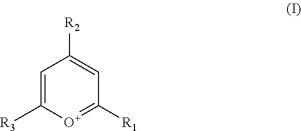

wherein R₁, R₂ and R₃ are the same or different and each is hydrogen, halogen or alkyl, or a salt thereof, as a labeling compound.
[9] The kit of the above-mentioned [8], wherein the compound of the formula (I) is 2,4,6-trimethylpyrylium.
[10] The kit of the above-mentioned [9], wherein the combination of two or more kinds of stable isotopes comprises two or more kinds of compounds selected from the group consisting of Py0, Py1, Py2, Py3, Py4, Py5, Py6, Py7 and Py8 represented by the formula (III):

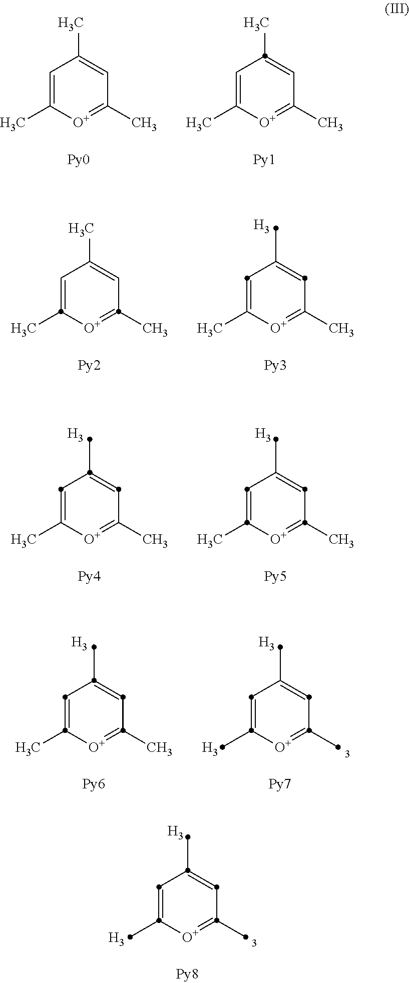

wherein carbon atoms shown by black balls have a mass number of 13, and salts thereof.
[11] A compound represented by the formula (II):

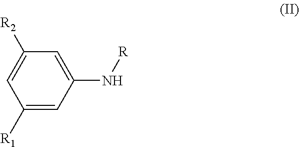

wherein R₁ and R₂ are the same or different and each is hydrogen, halogen or alkyl, and R is any optionally substituted hydrocarbon group,
which contains at least one carbon atom having a mass number of 13 at a position other than R in the formula (II), excluding a compound having a peptide bond, or a salt thereof.
[12] A compound represented by the formula (IV):

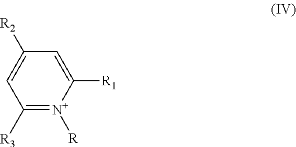

wherein R₁, R₂ and R₃ are the same or different and each is hydrogen, halogen or alkyl, and R is any optionally substituted hydrocarbon group,
which contains at least one carbon atom having a mass number of 13 at a position other than R in the formula (IV), excluding a compound having a peptide bond, or a salt thereof.

### Effect of the Invention

The quantitative method of the present invention enables quantification of an amino group-containing non-peptidic compound (e.g., biologically active amine such as neuroamine and the like, amino acid, stimulant etc.) contained at a low concentration (e.g., 0.01-0.1 picomolar) in a body fluid (blood, urine, cerebrospinal fluid etc.) or a biological tissue (brain etc.). Moreover, the analysis method of the present invention enables detection of the above-mentioned compound in many biological samples (e.g., 9 samples) all at once, and further, estimation of the structure thereof.

More specifically, for example, the analysis method of the present invention realizes highly sensitive and highly efficient multiple quantification for the elucidation of the cause of neuropsychiatric diseases and emotional disorders by the analysis of brain neurotransmitter amine (e.g., L-DOPA, dopamine, noradrenaline, serotonin, histamine etc.) or amino acid (e.g., glutamic acid, glycine, alanine, tryptophan etc.), clinical medicine and forensic examination by the analysis of biologically active amine, amino acid, stimulant or narcotic in blood, cerebrospinal fluid, lacrimal fluid or the like, and analysis of an amino group-containing non-peptidic compound in the environment or food, by detection and quantification of involatile putrefactive amine (e.g., histamine, tyramine, spermidine, spermine, putrescine, cadaverine etc.), which is a causative substance of an allergy-like food poisoning produced by a microbial action.

The kit and labeled product of the present invention are useful for performing the quantitative method of the present invention.

### BRIEF DESCRIPTION OF THE DRAWINGS

FIG. 1 is a schematic diagram showing the labeling of dopamine with Py compound (Py0-Py8).

FIG. 2 shows chemical formulas and molecular weights of derivatives labeled with Py compound (molecular weight when labeled with Py0) and the like, regarding various amino group-containing non-peptidic compounds.

FIG. 3 shows an ion chromatograph of mass M/z 258.1±0.1 in micro-LC separation.

FIG. 4 shows a mass spectrum data corresponding to peak 1 (resultant product 1) in the ion chromatograph of FIG. 3.

FIG. 5 shows a mass spectrum data corresponding to peak 2 (resultant product 2) in the ion chromatograph of FIG. 3.

FIG. 6-1 shows ion chromatographs of L-alanine, L-glutamic acid, glycine, GABA and histamine in micro-LC separation.

FIG. 6-2 shows ion chromatographs of ornithine, dopamine, noradrenaline, L-DOPA and serotonin in micro-LC separation.

FIG. 7 shows an ion chromatograph of mass M/z 258.1±0.1 in nano-LC separation.

FIG. 8 shows a mass spectrum of arrow (retention time: 32.9 min) in the ion chromatograph of FIG. 7.

FIG. 9 shows a mass spectrum of a portion corresponding to Wz=244.1 in nano-LC separation.

FIG. 10 shows a mass spectrum obtained by an experiment to confirm the detection sensitivity for dopamine by using 5 kinds of Py compounds, wherein 1, 2, 3, 4 and 5 correspond to dopamine labeled with Py0, Py2, Py4, Py6 and Py8, respectively.

FIG. 11 shows a graph plotting the intensities of mass spectral peaks 1 to 5 in FIG. 10 to the initial level of dopamin in each corresponding sample.

FIG. 12 shows the position for each sample of the brain section in the catecholamine quantification experiment in rat brain, wherein the numbers affixed to the image correspond to the kind of reacted Py reagent and 0 is Py0, 2 is Py2, 4 is Py4, 6 is Py6, and 8 is Py8.

FIG. 13 shows a nanospectrum data obtained by the catecholamine quantification experiment in rat brain, wherein the arrows show the corresponding relationship between the labeling reagent used and the peak.

FIG. 14 is a graph showing the calculated dopamine level at each region of the brain, based on the spectral intensity obtained in FIG. 13.

### DESCRIPTION OF EMBODIMENTS

### (Quantitative Method)

The quantitative method of the present invention is explained in detail in the following.

The quantitative method of the present invention enables determination of the absolute quantity of an amino group-containing non-peptidic compound to be measured, which is contained in one or more biological samples to be analyzed.

The number of biological samples to be analyzed is not particularly limited as long as the amino group-containing non-peptidic compound contained in each biological sample can be labeled with a combination of stable isotopes to produce mass difference. For example, when a combination of stable isotopes of the below-mentioned 2,4,6-trimethylpyrylium is used as a labeling compound, the maximum number of 8 biological samples can be analyzed at once besides the sample to be used as an internal standard sample.

Moreover, since the quantitative method of the present invention enables determination of the absolute quantity of an amino group-containing non-peptidic compound to be measured in a biological sample, analysis is possible by repeating a similar procedure, no matter how high the number of the target biological sample is.

The derivation and kind of the biological samples to be analyzed are not particularly limited, and the samples can be obtained from any derivation and tissues according to the analysis object. Specifically, examples of the biological sample include, but are not limited to, samples containing various body fluids (e.g., blood, bone marrow fluid, cerebrospinal fluid, saliva, lacrimal fluid, gastric fluid, ascites, exudate, amniotic membrane fluid, pancreatic juice, bile and the like), excretions (e.g., urine, stool and the like), and tissues (e.g., brain, spinal cord, eyeball, stomach, pancreas, kidney, liver, gonad, thyroid gland, gall bladder, bone marrow, adrenal gland, skin, lung, gastrointestinal tract (e.g., large intestine, small intestine), blood vessel, heart, thymus, spleen, submandibular gland, peripheral blood, prostate, orchis, ovary, placenta, uterus, bone, articular, adipose tissue, skeletal muscle and the like) and the like of mammals (e.g., human, monkey, bovine, horse, swine, sheep, goat, dog, cat, rabbit, hamster, guinea pig, mouse, rat etc.).

In the present specification, the amino group-containing non-peptidic compound or the non-peptidic compound having an amino group means any compound having one or more amino groups in a molecule, and free of a peptide bond in a molecule. Here, the amino group means a monovalent functional group obtained by removing a hydrogen from ammonia, primary amine (i.e., compound wherein one hydrogen atom of ammonia is substituted by any optionally substituted hydrocarbon group) or secondary amine (i.e., compound wherein two hydrogen atoms of ammonia are substituted by the same or different, any optionally substituted hydrocarbon groups). Thus, the amino group-containing non-peptidic compound is a non-peptidic compound having a chemical formula of NH₃, NH₂R, or NHRR′ wherein R and R′ are the same or different and each is any optionally substituted hydrocarbon group. However, a compound having a chemical formula of NHRR′ is considered to be unreactive or extremely low-reactive with the labeling reagent used in the method of the present invention. Therefore, the amino group-containing non-peptidic compound to be the target of the method of the present invention is generally a non-peptidic compound having a chemical formula of NH₂R wherein R is hydrogen or any optionally substituted hydrocarbon group.

While the molecular weight of the amino group-containing non-peptidic compound to be the measurement target is not particularly limited as long as the quantitative method of the present invention can be performed, it is generally a low molecular weight compound. A specific molecular weight is 17-1000, preferably 17-700, more preferably 17-500. Examples of the amino group-containing non-peptidic compound to be measured include biologically active amines, amino acids, drugs, stimulant drugs, narcotics, involatile putrefactive amines, and metabolites thereof having an amino group and the like. It is also possible to measure plural kinds of amino group-containing non-peptidic compounds by a single analysis.

More specific examples of the amino group-containing non-peptidic compound include, but are not limited to, biologically active amines that act on the nerve system (e.g., L-DOPA, norepinephrine, dopamine, tryptamine, serotonin, ptomaine, histamine, tyramine, taurine etc.), various biological amino acids (e.g., arginine, asparagine, aspartic acid, cysteine, glutamine, glutamic acid, glycine, histidine, isoleucine, leucine, lysine, methionine, phenylalanine, serine, threonine, tryptophan, tyrosine, valine, γ-aminobutyric acid (GABA), and modified products thereof (e.g., phosphorylated product etc.) etc.), drugs and narcotics (e.g., phenethylamine, amphetamine, cathine, cathinone, phentermine, mescaline, MDA, methoxyamphetamine, BDB, HMA, 2C-B, DOB, DOM, DOET, MMDA, TMA, 2C-I, 2C-D, 2C-N, 2C-T-2, 2C-T-7, DOI, DON, 2,5-DMA, 3,4-DMA etc.), involatile putrefactive amines (e.g., spermidine, spermine, putrescine, cadaverine etc.), as well as metabolites thereof having an amino group and the like.

In the above-mentioned step 1, one or more biological samples to be the analysis target, and an internal standard sample containing a known quantity of the amino group-containing non-peptidic compound to be measured are prepared.

The biological sample to be the analysis target can be harvested by a method known per se from the aforementioned derivation and tissue etc. Then, the amino group-containing non-peptidic compound to be measured is preferably concentrated in each biological sample by a suitable means such as solid phase extraction and the like. The concentration can be performed by, for example, the following procedures. That is, in each harvested biological sample, the amino group-containing non-peptidic compound to be measured and proteins are separated by a deproteinization treatment using a suitable means such as acid extraction and the like. Then, the amino group-containing non-peptidic compound to be measured in the deproteinized liquid sample is trapped by a cation exchange resin capable of selectively trapping same. Then, an acidic or neutral low molecular substance that non-specifically adsorbed to the resin is washed with alcohol. The residual resin-bound cation is eluted with hydrochloric acid and the like and thereafter hydrochloric acid is removed under reduced pressure. As a result, a biological sample to be analyzed can be prepared.

In the present specification, the internal standard sample refers to a sample containing an amino group-containing non-peptidic compound to be measured at a known concentration, which is subjected to a treatment similar to that for a sample to be analyzed, and can be utilized to determine the absolute quantity of the compound contained in each sample to be analyzed.

An internal standard sample can be prepared by, for example, dissolving a commercially available product of a compound to be measured in 0.05M HCl. While the concentration of the amino group-containing non-peptidic compound to be measured in the internal standard sample is not particularly limited, it is generally preferably near the assumed concentration of the compound in a sample to be analyzed.

In the above-mentioned step 2, a compound indicative of mixing ratio is added to each of one or more biological samples and internal standard sample to a known concentration. When used in the present specification, the “compound indicative of mixing ratio” refers to a compound present in each sample at a known concentration, which can be utilized to determine the mixing ratio of a mixture prepared from the above-mentioned one or more biological samples and internal standard sample in subsequent step 4. While the detail is described later, the mixing ratio can be determined based on the determination of the quantitative ratio of the compound indicative of mixing ratio derived from each sample and present in the mixture. Therefore, the compound indicative of mixing ratio needs to be an amino group-containing non-peptidic compound absent in all of the one or more biological samples to be analyzed and the internal standard sample. Specific examples of the compound indicative of mixing ratio include dihydroxybenzylamine (DHBA) and the like. While the amount to be added of the compound indicative of mixing ratio is not particularly limited as long as the mass spectrometry is not adversely affected, it is added such that, for example, the concentration of the compound indicative of mixing ratio in each sample is 0.2-10 pmol, preferably 0.5-5 pmol, more preferably 1-3 pmol. Preferably, the compound indicative of mixing ratio is added such that the concentration thereof is same in all of the one or more biological samples and the internal standard sample.

In the above-mentioned step 3, an amino group-containing non-peptidic compound to be measured and a compound indicative of mixing ratio, which are contained in all of the above-mentioned one or more biological samples and the internal standard sample, are labeled with an amino reactive reagent. In the quantitative method of the present invention, a combination of two or more kinds of stable isotopes of a compound represented by the formula (I):

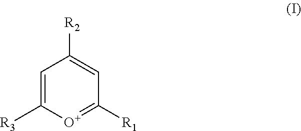

wherein R₁, R₂ and R₃ are the same or different and each is hydrogen, halogen or alkyl, or a salt thereof is used as a labeling compound.

In the formula (I), R₁, R₂ and R₃ are the same or different and each is hydrogen, halogen or alkyl. R₁, R₂ and R₃ are each preferably hydrogen, halogen, or alkyl having a carbon number of 1-6 (e.g., methyl, ethyl, propyl, isopropyl, butyl, sec-butyl, isobutyl, tert-butyl, pentyl, hexyl etc.), more preferably alkyl having a carbon number of 1-3 (e.g., methyl or ethyl). Examples of the aforementioned halogen include fluorine, chlorine, bromine, iodine and the like.

Preferable examples of the compound of the formula (I) include 2,4,6-trimethylpyrylium, 2-ethyl-4,6-dimethyl pyrylium, 2,6-diethyl-4-methylpyrylium and the like, and particularly preferred is 2,4,6-trimethylpyrylium.

In the quantitative method of the present invention, the compound of the formula (I) is generally used in the form of a salt. In this case, the salt consists of the compound of the formula (I) and any anion atom or anion molecule. Examples of the anion atom or anion molecule include anions such as an anion from hexafluorophosphoric acid, trifluoromethanesulfonic acid, tetrafluoroboric acid or the like. While the kind thereof is not subject to any particular limitation as long as it does not inhibit the labeling reaction of amino group-containing non-peptidic compound, it is preferably an anion from tetrafluoroboric acid.

Therefore, preferable examples of amino reactive reagent include 2,4,6-trimethylpyrylium tetrafluoroborate, 2-ethyl-4,6-dimethylpyrylium tetrafluoroborate, 2,6-diethyl-4-methyl pyrylium tetrafluoroborate and the like, particularly preferably, 2,4,6-trimethylpyrylium tetrafluoroborate.

The labeling in the above-mentioned step 3, which uses the above-mentioned amino reactive reagent having a mass difference due to stable isotopes produces a difference in the mass of the amino group-containing non-peptidic compound to be measured between samples as well as in the mass of the compound indicative of mixing ratio between samples. The mass difference between stable isotopes to be used is not particularly limited as long as the same kind of amino group-containing non-peptidic compound having a mass difference can be separated by a mass spectrometer. The mass difference is not less than 1 and only stable isotopes having a mass difference of not less than 2 may be selected and used in some cases. The upper limit of the mass difference is not particularly limited as long as amino reactive reagent can exist stably. Generally, since the mass difference between compounds is produced by a mass difference between ¹²C and ¹³C, the upper limit of the mass difference is the same as the number of carbon atoms contained in amino reactive reagent.

As mentioned above, preferable example of the compound of the formula (I) to be used for the quantitative method of the present invention includes 2,4,6-trimethylpyrylium. The 2,4,6-trimethylpyrylium is a compound having the following formula:

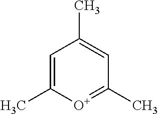

and contains 8 carbon atoms. In the stable isotopes to be used as the labeling compound in the present invention, the number of carbon isotope ¹³C may be any of 0 to 8, and a specific attention is paid to the position thereof according to the number of ¹³C. That is, the position of ¹³C is symmetrically arranged with respect to the line passing through the 1st position oxygen and the 4th position carbon.

Particularly preferable examples of the amino reactive reagent to be used for the quantitative method of the present invention include a combination of 9 kinds of stable isotopes of 2,4,6-trimethylpyrylium having a mass different by one, as shown below.

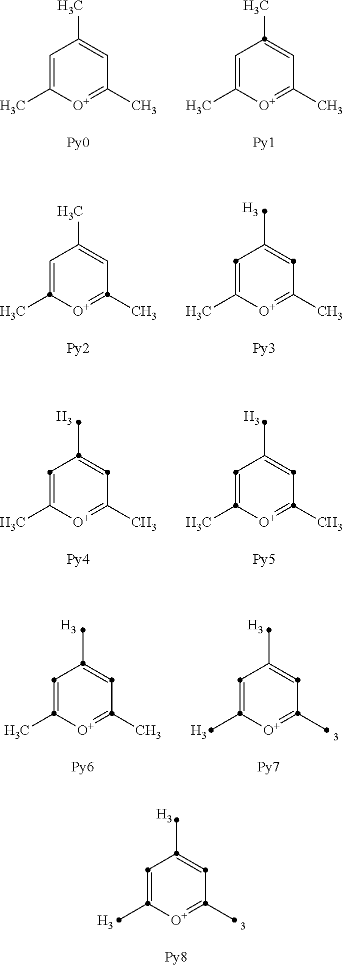

wherein carbon atoms shown by black ball has a carbon atom having a mass number of 13. Hereinafter, these compounds are collectively referred to as Py compound, and each stable isotope included in the Py compound is referred to as Py0, Py1, Py2, Py3, Py4, Py5, Py6, Py7 and Py8, based on the number of ¹³C in a molecule. For example, a tetrafluoroboric acid salt of the above-mentioned Py compound can be preferably used in the quantitative method of the present invention. In addition, any two kinds or more (e.g., 2 kinds, 3 kinds, 4 kinds, 5 kinds, 6 kinds, 7 kinds, 8 kinds, 9 kinds) selected from the above-mentioned 9 kinds of compounds may be combined and used in the quantitative method of the present invention.

The above-mentioned amino reactive reagent can be synthesized according to the methods taught in, for example,
1) Balaban, A. T., Boulton A. J., Organic Synthesis, Coll., vol. 5, p. 1112 (1973); vol. 49, p. 121 (1969).
2) Balaban, A. T., Boulton A. J., Organic Synthesis, Coll., vol. 5, p. 1114 (1973)
3) Ghiviriga I., Czerwinski E. W., Balaban A. T., Croatia Chemica Acta, vol. 77(1-2), p. 391-396 (2004).

Labeling with the above-mentioned Py compound can be performed as follows. That is, since a combination of 9 kinds of stable isotopes are available for Py compound, one kind of stable isotope is used for an internal standard sample, and any of the other 8 kinds is allocated to 1 to 8 biological samples to be analyzed. An amino group-containing non-peptidic compound contained in a different biological sample is labeled with a stable isotope having a different mass. Before labeling reaction, each biological sample is preferably adjusted to a suitable pH (e.g., pH 8.5-pH 11, preferably pH 9-pH 10) in advance. Then, each labeling reagent is added to each sample, and reacted at 25° C.-60° C., e.g., 50° C., for a suitable time (e.g., 10 min-180 min, for example, 30 min). After completion of the reaction, for example, hydrochloric acid is added to form acidic conditions to quench the reaction.

Py compound labels an amino group-containing non-peptidic compound according to the following reaction pathway (see, for example, C. Toma and Balaban A. T., Tetrahedron, Vol. 22 supplement No. 7 p. 9-22 (1966)). A labeling compound other than Py compound, which is shown by the above-mentioned formula (I), also labels an amino group-containing non-peptidic compound according to a similar pathway.

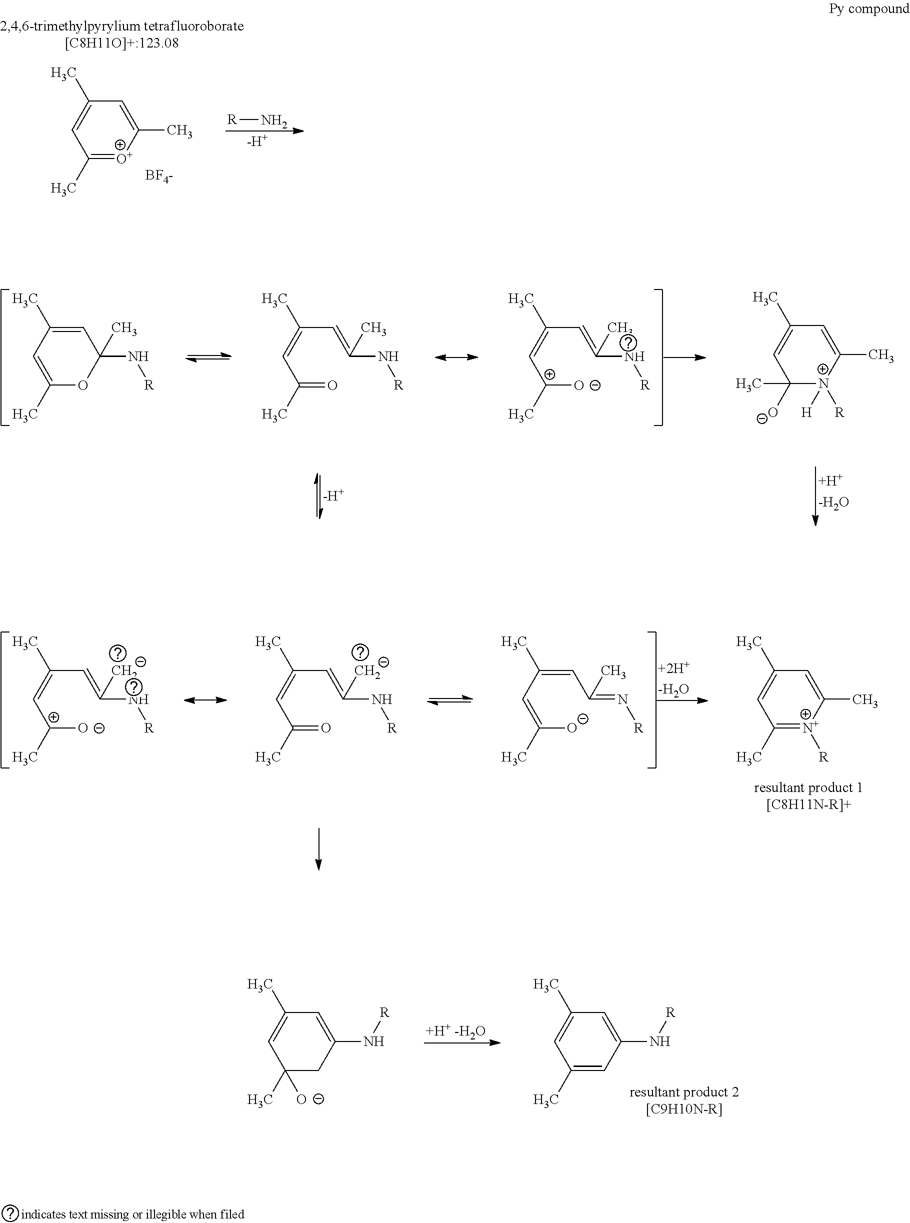

As shown in the above-mentioned reaction pathway, labeling produces two kinds of labeled products (resultant product 1 and resultant product 2) having a molecular weight different from each other. The presence ratio of resultant product 1 and resultant product 2 varies depending on the kind of an amino group-containing non-peptidic compound and reaction conditions. These two kinds of labeled products can be easily separated by a subsequent liquid chromatographic analysis.

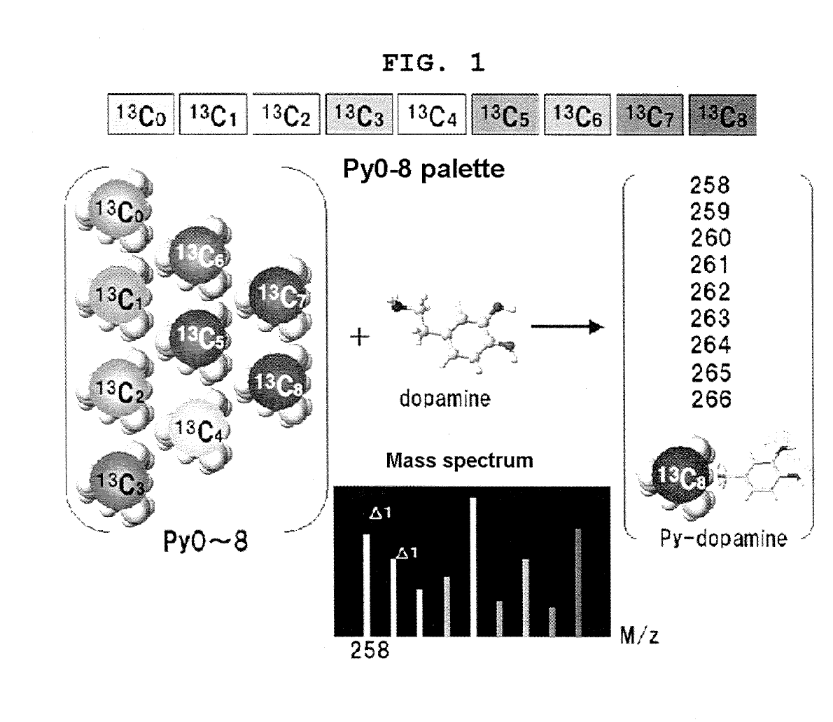

As a specific example, FIG. 1 shows an example wherein dopamine is labeled with Py compound to produce a difference in the mass. When dopamine is labeled with each of Py0, Py1, Py2, Py3, Py4, Py5, Py6, Py7 and Py8 compounds, Py-labeled dopamines having a molecular weight of about 258, 259, 260, 261, 262, 263, 264, 265, 266 or 267 are obtained.

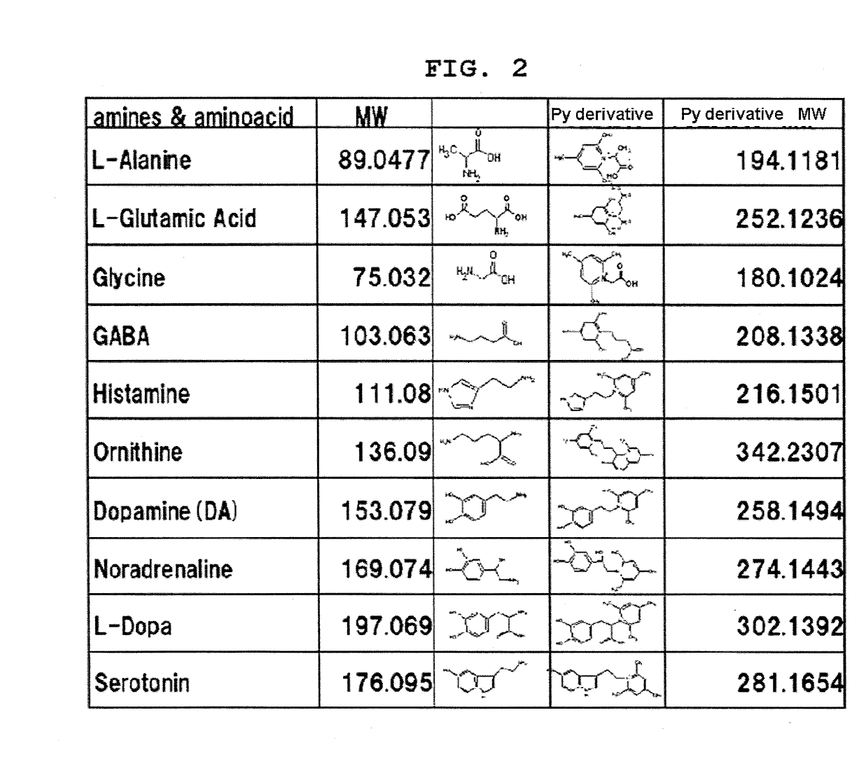

In addition, FIG. 2 shows the chemical formulas and molecular weights of the derivatives of some amines and amino acids which are labeled with a Py compound (specifically, Py0 compound).

In the above-mentioned step 4, a given amount is taken from each of the above-mentioned one or more biological samples and the internal standard sample, which have been labeled with an amino reactive reagent having a mass difference, and they are mixed to give a mixture. The amount taken from each sample for the preparation of the mixture is preferably equal, but is not limited thereto. An excess labeling reagent present in the mixture may be removed, though the removal is not always necessary. An excess labeling reagent can be removed by organic solvent extraction using ethyl acetate and the like, or by using a cation exchange resin. Furthermore, the mixture is preferably concentrated before mass spectrometry. For concentration of the mixture, the mixture is added to an equilibrated cation exchange resin (H⁺-type), washed well with water, and the reaction product is eluted with 1% aqueous ammonia or 0.1M hydrochloric acid. The elution is performed by concentration to a suitable amount by a reduced pressure centrifugal concentrator.

In the above-mentioned step 5, the mixture prepared according to the above-mentioned procedures is subjected to mass spectrometry. Mass spectrometry can be performed according to a known method. While the quantitative method of the present invention permits use of any mass spectrometry system, analysis using a nano-liquid chromatographic mass spectrometry (nano-LC/MS) system is preferable since it enables high sensitive quantification. Examples of the usable nano-LC/MS apparatus include NanoFrontier eLD (manufactured by Hitachi High-Technologies Corporation) and the like. Mass spectrometry can be performed, for example, specifically as follows. That is, using monolith-type MonoCap for FastFlow (0.075 mm ID×150 mL, Merck & Co., Inc.) as a separation column, and C18-Monolith trap column (0.05 mm ID×150 mm L Hitachi) as a trap column, gradient elution is performed at flow 200 nl/min, mobile phase A) formic acid/water/acetonitrile (0.1:98:2), B) formic acid/water/acetonitrile (0.1:2:98) (i.e., A/B=98/2 (0 min)-50/50 (50 min)-0/100 (50.1-70 min)-98/2 (70.1-90 min)). The sample injection volume is 50 nl. Mass section settings: ionization mode nano-ESI (positive ionization), spray voltage 1400 V, detector voltage 2150 V, counter nitrogen gas rate 0.8 L/min, scan range 50-1000 m/z. The mass spectrometry results are recorded for 50 min.

Due to the isotope labeling, the same kind of amino group-containing non-peptidic compounds derived from different samples have different mass. Therefore, in the mass spectrum obtained by mass spectrometry, the amino group-containing non-peptidic compounds derived from different samples appear as separated peaks. In this way, the intensities of peaks, which correspond to the masses of the amino group-containing non-peptidic compounds to be measured having a mass difference each other, and the intensities of peaks, which correspond to the masses of the compounds indicative of mixing ratio having a mass difference from each other can be obtained. In the following, two or more peaks corresponding to the same compound except the mass difference due to isotope labeling are also referred to as a peak group. As mentioned above, since two labeled products different from each other in the molecular weight and chemical property (i.e., the above-mentioned resultant product 1 and resultant product 2) can be obtained for each compound, two kinds of peak groups can be obtained for each compound. In the following steps, the intensity is compared between two or more peaks included in same peak group. Since the amounts of resultant product 1 and resultant product 2 formed vary depending on the kind of the amino group-containing non-peptidic compound, the intensity is preferably compared in the peak group of the resultant product with high spectrum intensity.

In the above-mentioned step 6 and step 7, the absolute quantity of an amino group-containing non-peptidic compound to be measured, which is contained in each biological sample to be analyzed, is determined from the peak group obtained as mentioned above.

First, the relative amount between respective samples of an amino group-containing non-peptidic compound to be measured is determined in step 6. Therefor, the intensity ratio of the peak corresponding to the compound in each of the biological samples to be analyzed, and the peak corresponding to the compound in internal standard sample is obtained. The intensity ratio corresponds to the quantitative ratio of the compound with mass difference due to isotope labeling, which is contained in the mixture subjected to mass spectrometry. Therefore, the quantitative ratio of the compound in each sample in the mixture can be determined based on the intensity ratio.

Subsequently in step 7, a mixing ratio of one or more biological samples to be analyzed and the internal standard sample is determined for the preparation of the above-mentioned mixture. As mentioned above, since the compound indicative of mixing ratio is added to each sample before mixing to a known concentration, the mixing ratio can be determined based on comparison of the intensities of the peak group corresponding to the compound indicative of mixing ratio. Furthermore, by utilizing the mixing ratio and the quantitative ratio of the compound in each sample, which is determined in the above-mentioned step 6, an absolute quantity of the compound contained in one or more biological samples to be analyzed can be determined.

### (Quantification Kit)

The present invention also provides a reagent kit usable for the aforementioned quantitative method of the amino group-containing non-peptidic compound, which comprises, as a labeling compound, a combination of two or more kinds of stable isotopes of a compound represented by the formula (I) or a salt thereof (hereinafter to be also referred to as the kit of the present invention). The definitions relating to the to compound represented by the formula (I) or a salt thereof, stable isotopes and embodiment of combination are as mentioned above.

The kit of the present invention may contain, besides the is aforementioned combination of stable isotopes, one or more kinds of reaction buffers, wash solutions, or other components necessary or preferable for the combined use with labeling reagent in the present invention. Also, the kit of the present invention optionally contains an instruction manual. Moreover, the kit of the present invention may further contain a reagent for removing unreacted components (wash reagent), a restriction enzyme, a column for purification, a purification solvent and the like.

### (Labeled Product)

The present invention further provides a compound represented by the following formula:

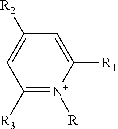

wherein R₁, R₂ and R₃ are the same or different and each is hydrogen, halogen or alkyl, and R is any optionally substituted hydrocarbon group,
which contains at least one carbon atom having a mass number of 13 at a position other than R in the formula, excluding a compound having a peptide bond (hereinafter to be also referred to as compound 1) and a salt thereof, and a compound represented by the following formula:

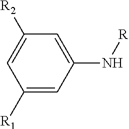

wherein R₁ and R₂ are the same or different and each is hydrogen, halogen or alkyl, R is any optionally substituted hydrocarbon group,
which contains at least one carbon atom having a mass number of 13 at a position other than R in the formula, excluding a compound having a peptide bond (hereinafter to be also referred to as compound 2) and a salt thereof (hereinafter these compounds are also collectively referred to as the labeled product of the present invention). The labeled product of the present invention can be utilized for, for example, preparation of an internal standard sample (wherein an amino group-containing non-peptidic compound to be analyzed is already labeled with a labeling reagent having ¹³C) for the quantitative method of the present invention.

The labeled product of the present invention may be produced, for example, by labeling an amino group-containing non-peptidic compound defined above with a labeling reagent (limited to one having ¹³C) to be used for the quantitative method of the present invention. That is, any compound obtained by labeling the amino group-containing non-peptidic compound defined above with said labeling reagent is encompassed in the labeled product of the present invention.

Thus, the definition of R₁, R₂ and R₃ in the formula of compound 1 or compound 2 is, the same as that mentioned above for R₁, R₂ and R₃ in the labeling compound used for the quantitative method of the present invention. Combinations thereof are also similar to those in the labeling compound. Examples thereof include a combination of R₁, R₂ and R₃ in compound 1 each being a methyl group, a combination of R₁ and R₂ in compound 2 each being a methyl group, and the like.

R in the formula of compound 1 or compound 2 may also be the same as R in any amino group-containing non-peptidic compound having a chemical formula of NH₂R wherein R is hydrogen or any optionally substituted hydrocarbon group, which is described above for the quantitative method of the present invention.

The salt of compound 1 or compound 2 may be any salt. For example, those exemplified as the salt of the compound of the formula (I) (e.g., hexafluorophosphate, trifluoromethanesulfonate, tetrafluoroboric acid salt etc.), nitrate, hydrochloride, nitrate, sulfate and the like can be mentioned.

The number of ¹³C is one or more, and any number not more than the total number of carbon atom at the position other than for R in the formula of compound 1 or compound 2. The position of ¹³C can be derived according to the position of ¹³C in the labeling reagent (mentioned above) and the labeling reaction thereof (mentioned above).

The present invention is explained in more detail in the following by referring to Examples and the like, which are not to be construed as limitative.

### EXAMPLES

### Experimental Example 1

### Analysis of Dopamine by Micro-LC/MS and Using 5 Kinds of Py Compounds Having Mass Difference 2 from Each Other

To 10 mM dopamine standard solution (dissolved in 0.05M hydrochloric acid solution, 1 μl) were added 50 mM sodium borate buffer (pH 10.0, 5 μl), 50 mM Py reagent (mixture of equal amounts of 50 mM Py0, Py2, Py4, Py6 and Py8, 1 μl) and distilled water (3 μl) to the total amount of 10 μl, and the mixture was incubated at 50° C. for 30 min. After completion, 1M hydrochloric acid (2 μl) was added to produce acidic conditions. The mixture (2 μl) was directly introduced into micro-LC/MS and MS analysis was carried out. LC conditions were Develosil C30-UG-3 2.0 mmID×150 mm L column of reversed-phase system, flow set to 200 μl/min. As a mobile phase, 0.1% aqueous formic acid solution as solution A and 0.1% formic acid-containing acetonitrile as solution B were used, and a density gradient elution method was adopted at A/B=10/90 (0 min)-0/100 (10-20 min)-10/90 (20.1 min). The column temperature was set 20 to 40° C. The MS measurement conditions were ionization mode of semimicro positive ionization ESI mode, nebulizer gas flow rate 3.0 L/min, AUX gas flow rate 12.0 L/min, and gas temperature 600° C. The voltage applied for spraying was 3500V, and the voltage applied to the detector was 2200V. The mass scan range was 50-500.

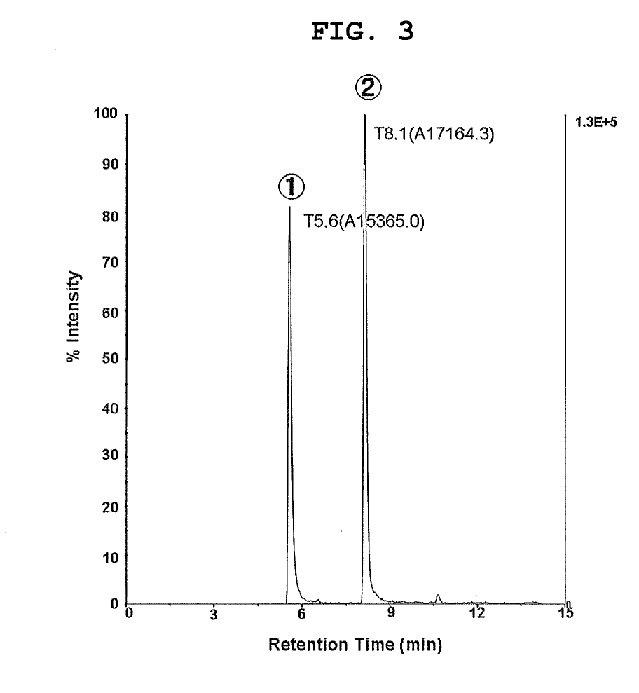

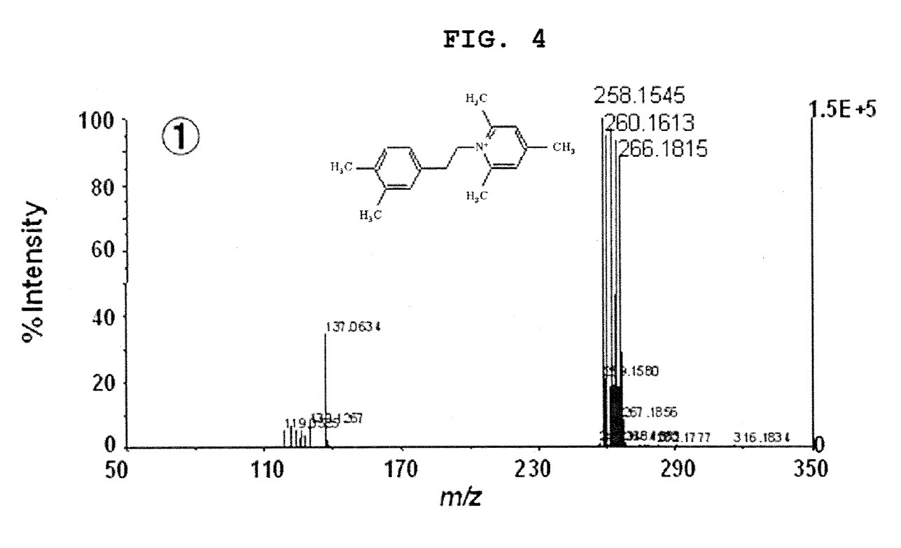

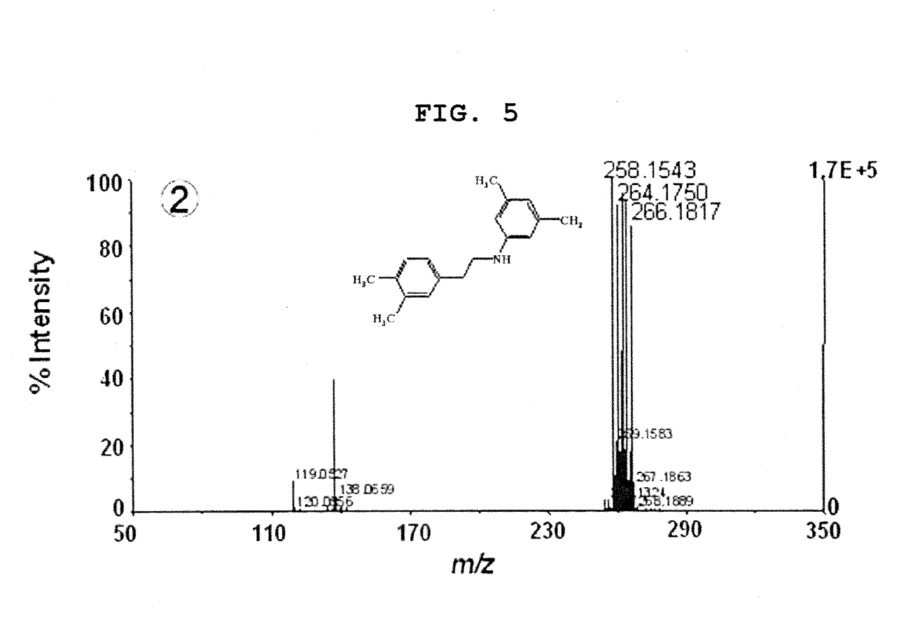

FIG. 3 shows an ion chromatograph of mass M/z 258.1±0.1 in LC separation, wherein two peaks are observed. They correspond to the resultant products 1 and 2. From the elution condition and property of pyridinium ion, peak 1 at 5.6 min corresponds to resultant product 1, and peak 2 at 8.1 min is estimated to be xylidine-type resultant product 2. While the mass of the both originally differs by only 1, since resultant product 2 is [M+H]⁺, it cannot be distinguished from resultant product 1. It is known the quantitative ratio of peaks 1 and 2 varies depending on the reaction conditions. FIG. 4 shows the mass spectrum of peak 1, and FIG. 5 shows the mass spectrum of peak 2. In any case, 5 peaks different in mass by 2 are observed from mass 244.1 with almost the same intensity.

### Experimental Example 2

### Analysis Examples of Plural Components of Amine and Amino Acid

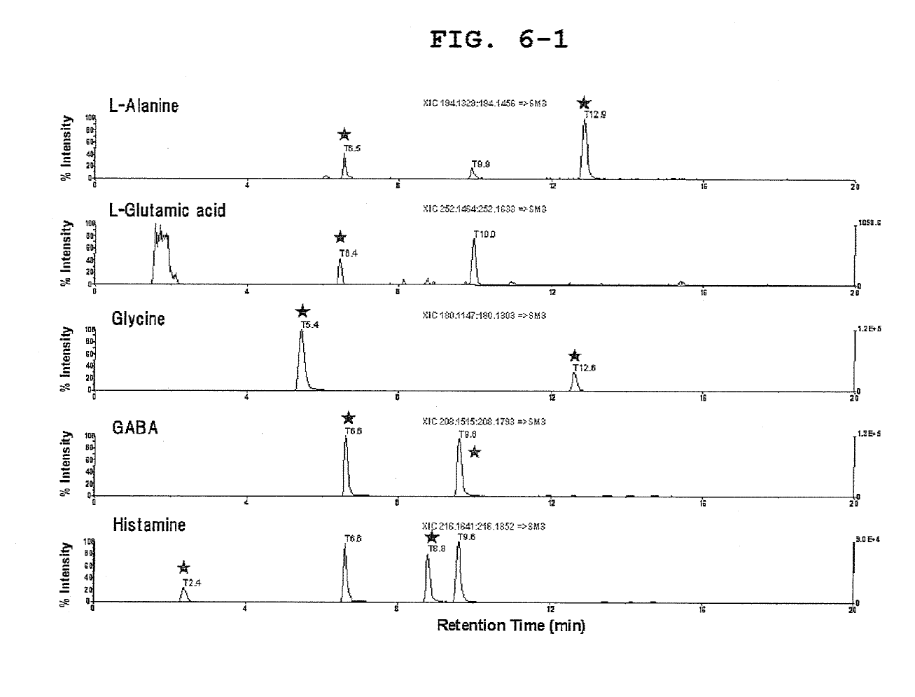

In the same manner as in the above-mentioned Experimental Example 1, amines and amino acids other than dopamine (FIG. 2) were individually analyzed by Py-derivatizing 10 mM standard solution of each amine or amino acid and performing micro-LC/MS. Ion chromatograph showing mass M/z value of each derivative is shown in FIG. 6.

Ten kinds of substances react with Py reagent to form derivatives, under the same conditions as in Experimental Example 1, and resultant product 1 and resultant product 2 were produced as shown by mark ★ in the Figure. In addition, it was also found that the elution time and quantitative production ratio thereof vary depending on the target substance, and the substances can be separated from each other.

### Experimental Example 3

### Analysis of Dopamine by Nano-LC/MS and Using 5 Kinds of Py Compounds Having Mass Difference 2 from Each Other

Using nano-LC/MS for high sensitive analysis, an experiment was carried out as follows.

The total amount (20 μl) of 10 mM dopamine standard solution (0.05M HCl, 2 μl), 10 mM DHBA (dihydroxybenzoic acid, 2 μl), 50 mM sodium borate buffer (pH 10.0, 10 μl), 25 mM Py reagent (mixture of equal amount of 5 kinds of Py0, Py2, Py4, Py6 and Py8, each 25 mM, 2 μl) and distilled water (4 μl) was incubated at 50° C. for 120 min. After cooling, 1M HCl (2 μl) was added to acidify the reaction mixture, which was diluted with 0.05M HCl and introduced by a manual injector into nano-LC/MS analysis. The nano-LC analysis conditions and MS measurement conditions are the same as those mentioned above.

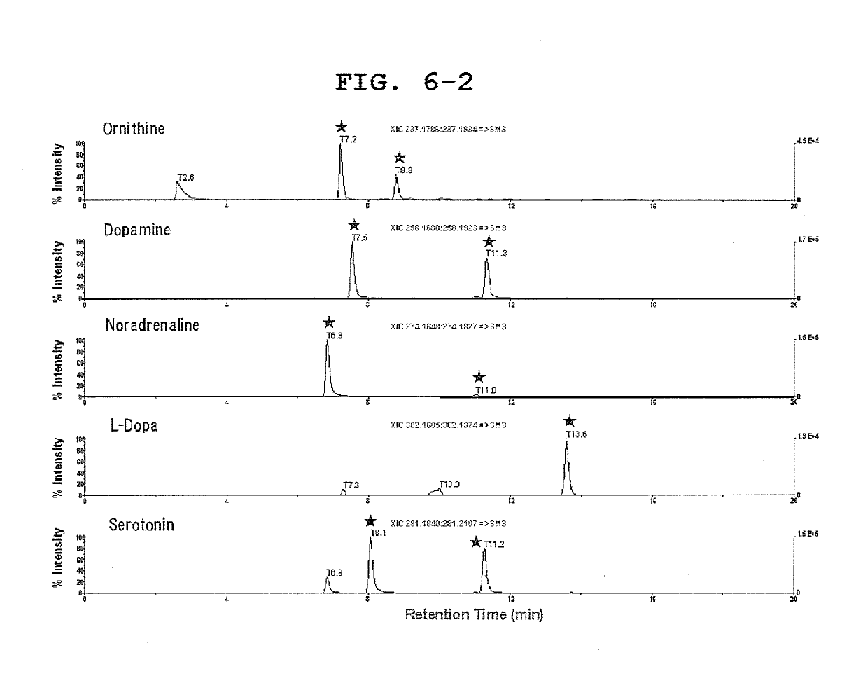

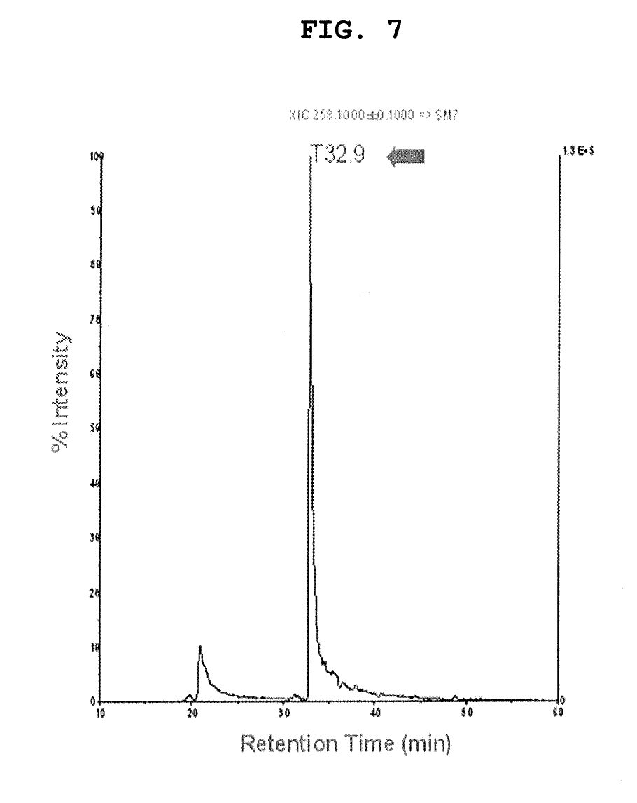

As shown in FIG. 7, a substance of mass M/z=258.1 was eluted at 32.9 min by nano-LC separation. The mass spectrum of the portion is shown in FIG. 8. Five peaks were detected every 2 Da, corresponding to dopamine labeled with Py reagent. These correspond to dopamines labeled with Py0, Py2, Py4, Py6 and Py8, respectively.

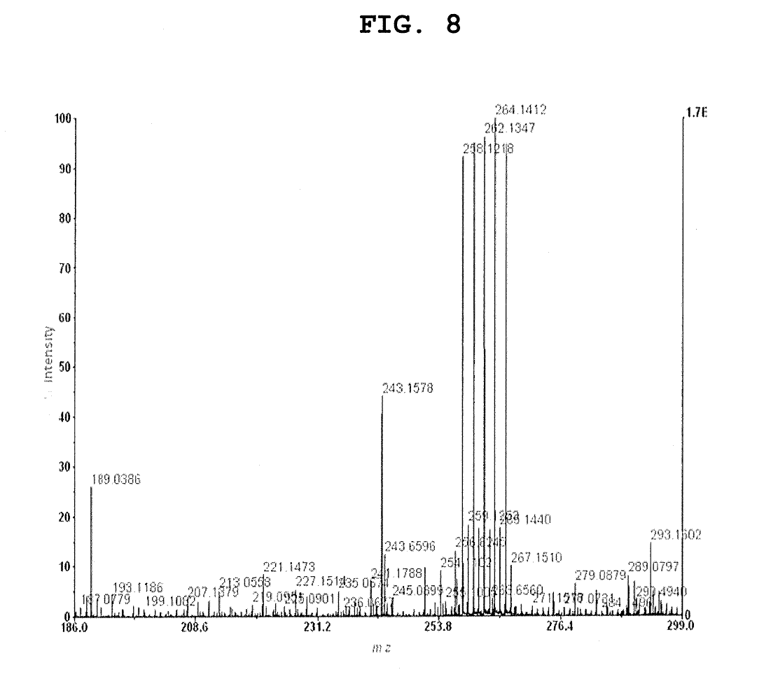

In addition, mass spectrometry at M/z=244.1 position showing elution at different time from dopamine was performed by the same chromatography. As a result, 5 peaks (black arrow tips) were observed at 2 Da difference from 244.1078, as shown in FIG. 9. This can be utilized as an internal standard for the amendment of recovery rate of operations such as solid phase purification of a reaction product of dopamine and Py reagent, and the like.

### Experimental Example 4

### Confirmation of Detection Sensitivity for Dopamine

Dopamine was taken in 5 tubes at 5.0 pmol, 1.25 pmol, 0.625 pmol, 0.3 pmol and 0.15 pmol, and the total amount of 8 μl was reached by adding 5% TCA. 2M phosphate K buffer (pH 11, 12 μl) was added, and the mixture was maintained at pH 10. 5 kinds of 100 mM Py reagents (1 μl) (Py0 corresponds to 5.0 pmol, Py2 to 1.25 pmol, Py4 to 0.625 pmol, Py6 to 0.3 pmol, Py8 to 0.15 pmol) were added, and the mixture was incubated at 50° C. for 5 min. The reaction was quenched by adding 6M hydrochloric acid (4 μl). The total amount was set to 25 μl. The mixture (20 μl) was harvested from each reaction tube and mixed. The mixture (100 μl) was treated with PBA (phenylboronic acid) resin (MonoSpinPBA column: GL Sciences Inc.), and a Py-derivatized compound having a catechol group was purified. That is, PBA column was activated with 2% TFA (trifluoroacetic acid)-containing 50% acetonitrile, and washed well with 100 mM potassium phosphate buffer (pH 8.0). To the above-mentioned mixture was added an equal amount of 1M potassium phosphate buffer (pH 10), and the mixture was stirred well and loaded on this column. The column was washed twice with acetonitrile and twice with 100 mM potassium phosphate (pH 8.0), and eluted with 2% TFA (trifluoroacetic acid)-containing 50% acetonitrile (30 μl). The eluate was concentrated to 10 μl, and 5 μl thereof was injected, and analyzed by nano-LC/MS. The analysis conditions were the same as those mentioned above.

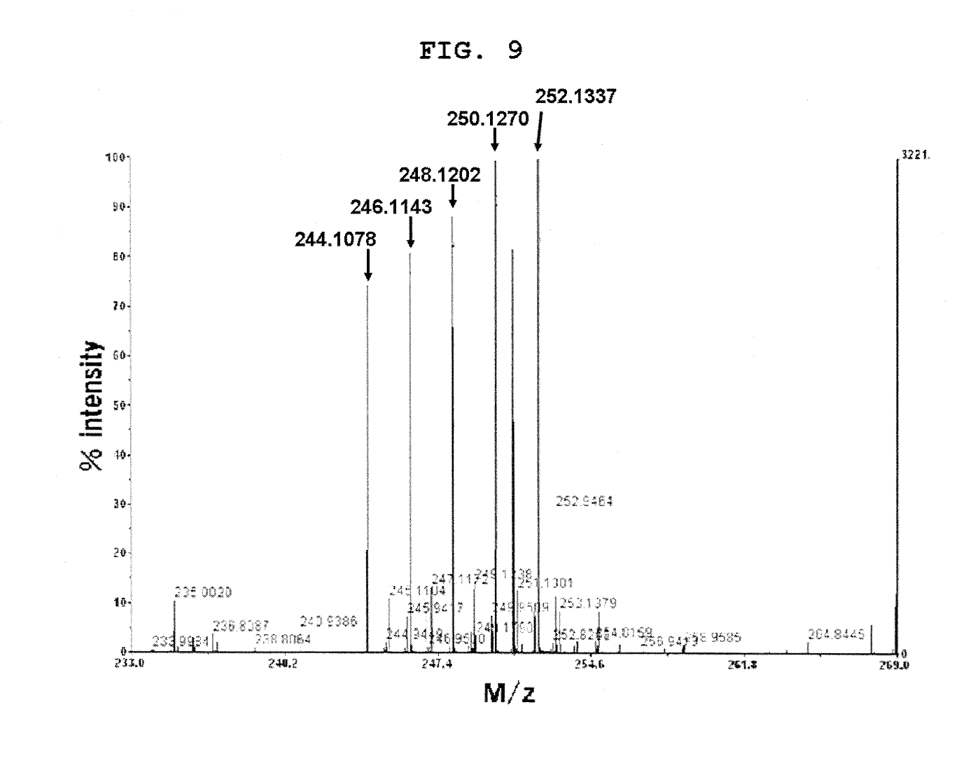

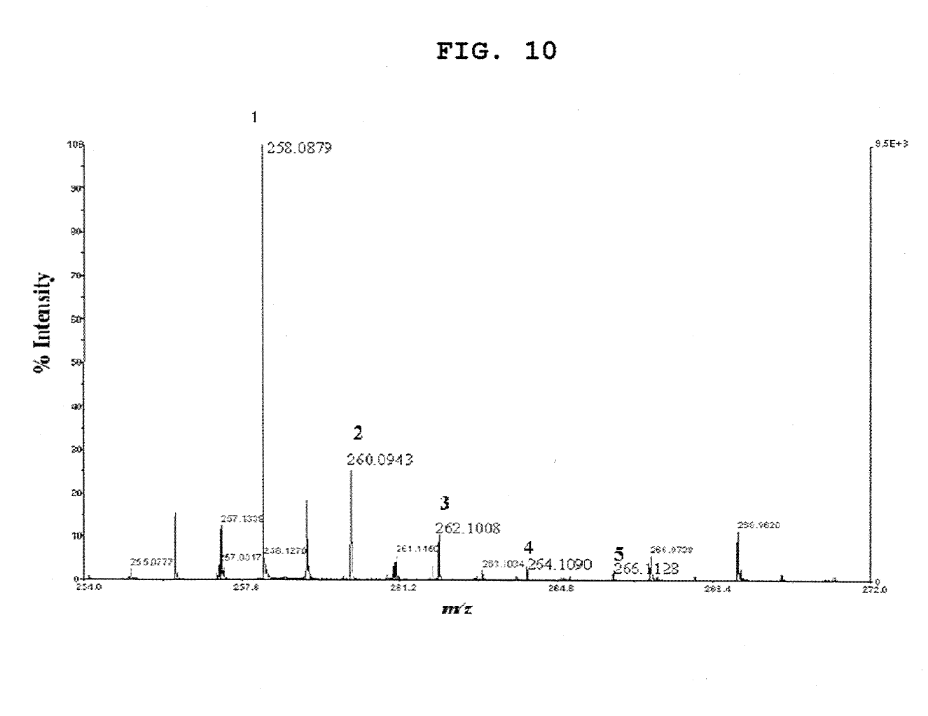

The mass of dopamine that reacted with Py0 (Py0-dopamine) was 258.0879, that of Py2-dopamine was 260.0943, that of Py-4-dopamine was 262.1008, that of Py6-dopamine was 264.1090, and that of Py8-dopamine was 266.1128. It was found that the mass difference was within the range of 2.0038-2.0080, and the peaks reflect the mass differences of Py reagents (FIG. 10). The peak intensities were plotted against the initial amount of dopamine and a linear relationship was obtained (FIG. 11). Thus, it was found that 0.15 pmol dopamine could be quantified.

### Example 1

### Quantification of Catecholamine in Rat Brain

An example of quantification of dopamine in rat brain minute tissue by the method of the present invention is shown below.

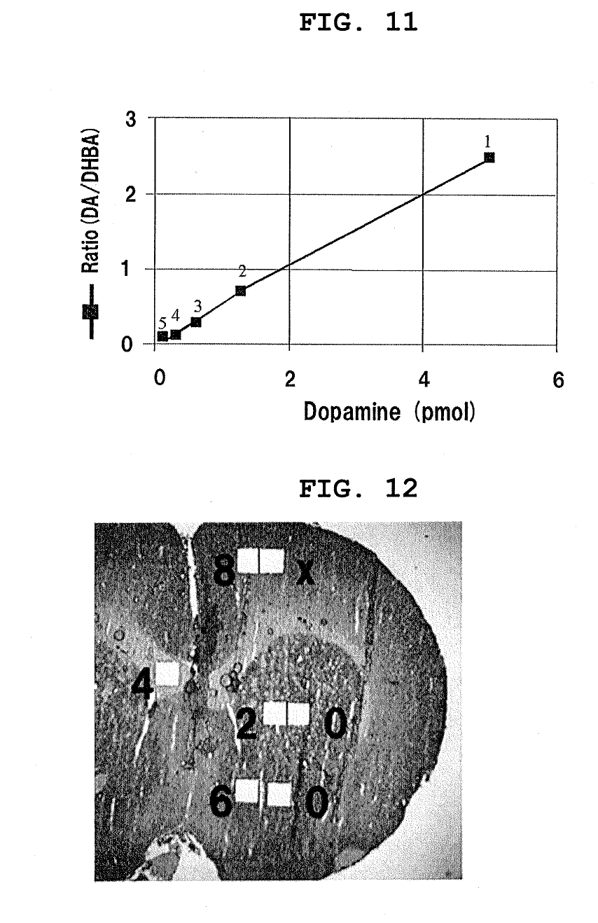

First, microwave was irradiated on rat head part (5 kW, 1.7 seconds) (Microwave Applicator, Muromachi Kikai Co., Ltd.), and the rat was fixed. The brain was removed, a 40 μm-thick brain tissue section was prepared using a freezing microtome (CM3050S, Leica), and 40 μm-thick, 500 μm-square brain tissues were obtained by a Laser Microdissection (ASLMD, Leica). In FIG. 12, the positions of the samples taken from various regions of the brain are shown white in the image of the brain section, wherein the numbers in the image indicate the kind of the Py reagents allowed to react (that is, 0, 2, 4, 6 and 8 correspond to Py0, Py2, Py4, Py6 and Py8, respectively). The correspondence between the kind of Py reagents and the brain region is shown in the following Table 1.

TABLE 1

| kind of Py reagent | Brain region (volume) |
|---|---|
| Py0 | striatum (10 nl × 2) |
| Py2 | striatum (10 nl) |
| Py4 | corpus callosum (10 nl) |
| Py6 | cerebral fornix + striatum (10 nl) |
| Py8 | cerebral cortex (10 nl) |

Each harvested section is 0.5 mm square and 40 μm thick, and therefore, the volume corresponds to 0.01 μl.

Then, DHBA was added as an internal standard substance at 2 pmol per sample, and a deproteinization treatment was further carried out by acid extraction method. Thereafter, Py compound was reacted with dopamine in the sample (reaction conditions: 50° C., 5 min), and the reaction was quenched by the addition of hydrochloric acid.

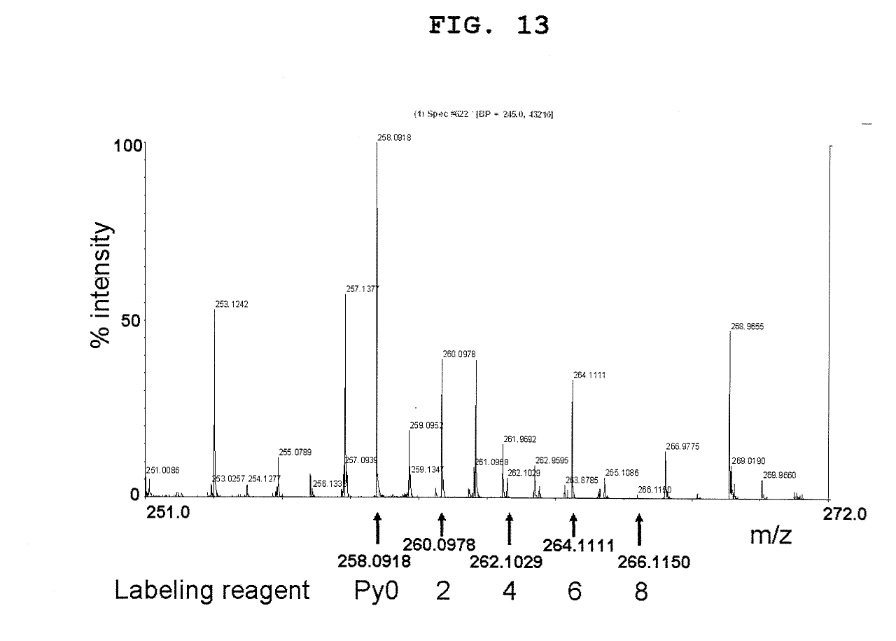

Using a column having phenylboric acid (PBA) as a functional group (MonoSpin PBA, GL Sciences Inc.), dopamine-Py compound in the sample was purified. A sample containing dopamine-Py compound was adjusted to pH 8-9 with an alkaline solution (aqueous dibasic potassium phosphate solution). The dopamine-Py compound was bound to PBA, washed with acetonitrile and water, and eluted with 2% trifluoroacetic acid-containing 50% acetonitrile solution. As mentioned above in the Description of Embodiments, the eluate was concentrated by a reduced pressure centrifugal concentrator and analyzed by a nano-LC/MS system. The obtained nanospectral data are shown in FIG. 13.

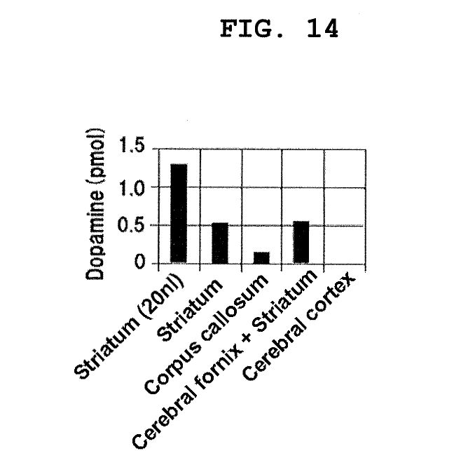

The amount of dopamine in each region was calculated from the obtained intensity, which is shown in FIG. 14. By measurement of dopamine in two sheets of striatum (10 nl, total 20 nl) with Py0, the value of about 1.3 pmol was obtained. By measurement of dopamine in striatum (10 nl) with Py2 and Py6, the value of about 0.5 pmol was obtained. By measurement of dopamine in corpus callosum (10 nl) with Py4, the value of about 0.2 pmol was obtained. In addition, by measurement of dopamine in cerebral cortex (10 nl) with Py8, the result was below detection sensitivity.

From these results, it was continued that measurement of dopamine concentration in brain tissue (10 nl) is possible by combining purification of dopamine-Py compound by MonoSpin PBA and a measurement method using Py reagent according to the method of the present invention.

### INDUSTRIAL APPLICABILITY

The quantitative method of the present invention enables quantification of an amino group-containing non-peptidic compound (e.g., biologically active amine such as neuroamine and the like, amino acid, stimulant etc.) contained at a low concentration (e.g., 0.01-0.1 picomolar) in a body fluid (blood, urine, cerebrospinal fluid etc.) or a biological tissue (brain etc.). Moreover, the analysis method of the present invention enables detection of the above-mentioned compound in many biological samples (e.g., 9 samples) all at once, and further, estimation of the structure thereof.

More specifically, for example, the analysis method of the present invention realizes highly sensitive and highly efficient multiple quantification for the elucidation of the cause of neuropsychiatric diseases and emotional disorders by the analysis of brain neurotransmitter amine (e.g., L-DOPA, dopamine, noradrenaline, serotonin, histamine etc.) or amino acid (e.g., glutamic acid, glycine, alanine, tryptophan etc.), clinical medicine and forensic examination by the analysis of biologically active amine, amino acid, stimulant or narcotic in blood, cerebrospinal fluid, lacrimal fluid or the like, and analysis of an amino group-containing non-peptidic compound in the environment or food, by detection and quantification of involatile putrefactive amine (e.g., histamine, tyramine, spermidine, spermine, putrescine, cadaverine etc.), which is a causative substance of an allergy-like food poisoning produced by a microbial action.

The kit and labeled product of the present invention are useful for performing the quantitative method of the present invention.

This application is based on patent application Nos. 2011-080943 (filing date: Mar. 31, 2011) and 2012-036598 (filing date: Feb. 22, 2012) filed in Japan, the contents of which are incorporated in full herein.

1. A method of quantifying a target non-peptidic compound having an amino group contained in one or more biological samples, which comprises the following steps:
(step 1) a step of preparing one or more biological samples to be analyzed, and an internal standard sample containing a known quantity of the target non-peptidic compound;
(step 2) a step of adding, as a compound indicative of mixing ratio, an amino group-containing non-peptidic compound, which is absent in the one or more biological samples and the internal standard sample, to each of the one or more biological samples and the internal standard sample, to a known concentration;
(step 3) a step of producing a difference in the mass of the target non-peptidic compound between samples comprising the one or more biological samples and the internal standard sample, and in the mass of the compound indicative of mixing ratio between samples comprising the one or more biological samples and the internal standard sample, by using a combination of two or more kinds of stable isotopes of a compound represented by the formula (I): 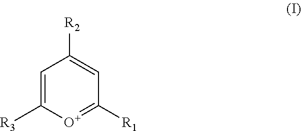
wherein R₁, R₂ and R₃ are the same or different and each is hydrogen, halogen or alkyl, or a salt thereof, as a labeling compound;
(step 4) a step of taking a given amount each from the one or more biological samples and the internal standard sample, and mixing them to give a mixture;
(step 5) a step of subjecting the mixture to mass spectrometry, and obtaining the intensities of peaks in a mass spectrum, which correspond to the masses of the target non-peptidic compounds having a mass difference from each other, and the intensities of peaks in the mass spectrum, which correspond to the masses of the compounds indicative of mixing ratio having a mass difference from each other;
(step 6) a step of determining a quantitative ratio between the target non-peptidic compounds having a mass difference from each other, which are contained in the mixture, based on an intensity ratio of the intensity corresponding to each of the target non-peptidic compounds derived from each of the one or more biological samples, and the intensity corresponding to the target non-peptidic compound derived from the internal standard sample; and
(step 7) a step of determining an absolute quantity of the target non-peptidic compound contained in each of the one or more biological samples, based on the quantitative ratio determined in step 6 and a mixing ratio of the samples determined by comparison of the intensities of peaks in the mass spectrum, which correspond to the masses of the compounds indicative of mixing ratio having a mass difference from each other.

2. The method of claim 1, wherein the compound of the formula (I) is 2,4,6-trimethylpyrylium.

3. The method of claim 1, wherein at least one of the target non-peptidic compounds and the compounds indicative of mixing ratio, from which the peak intensity obtained in step 5 is derived, is a compound represented by the formula (II): 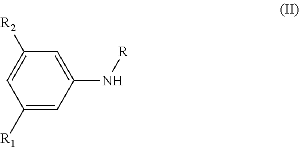
wherein R₁ and R₂ are as defined above, and R is any optionally substituted hydrocarbon group, excluding a compound having a peptide bond, or a salt thereof.

4. The method of claim 1, wherein the combination comprises 5 or more kinds of stable isotopes.

5. The method of claim 1, wherein the target non-peptidic compound is biologically active amine and/or amino acid.

6. The method of claim 5, wherein the biologically active amine is dopamine.

7. The method of claim 1, wherein the mass spectrometry is performed by a nano-liquid chromatographic mass spectrometer.

8. A kit for quantifying a target non-peptidic compound having an amino group contained in one or more biological samples, which comprises a combination of two or more kinds of stable isotopes of a compound represented by the formula (I): 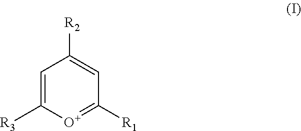
wherein R₁, R₂ and R₃ are the same or different and each is hydrogen, halogen or alkyl, or a salt thereof, as a labeling compound.

9. The kit of claim 8, wherein the compound of the formula (I) is 2,4,6-trimethylpyrylium.

10. The kit of claim 9, wherein the combination of two or more kinds of stable isotopes comprises two or more kinds of compounds selected from the group consisting of Py0, Py1, Py2, Py3, Py4, Py5, Py6, Py7 and Py8 represented by the formula (III): 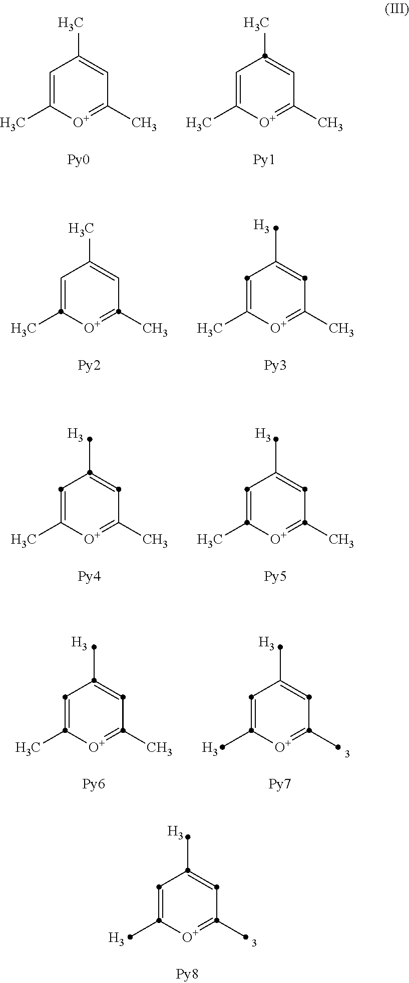
wherein carbon atoms shown by black balls have a mass number of 13, and salts thereof.

11. A compound represented by the formula (II): 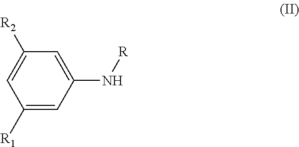
wherein R₁ and R₂ are the same or different and each is hydrogen, halogen or alkyl, and R is any optionally substituted hydrocarbon group,
which contains at least one carbon atom having a mass number of 13 at a position other than R in the formula (II),
excluding a compound having a peptide bond, or a salt thereof.

12. A compound represented by the formula (IV): 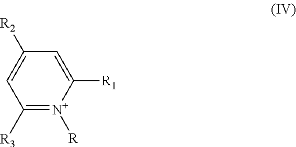
wherein R₁, R₂ and R₃ are the same or different and each is hydrogen, halogen or alkyl, and R is any optionally substituted hydrocarbon group,
which contains at least one carbon atom having a mass number of 13 at a position other than R in the formula (IV),
excluding a compound having a peptide bond, or a salt thereof.
